(19) United States
(12) Patent Application Publication
(10) Pub. No.: US 2026/0249046 A1
(43) Pub. Date: Aug. 27, 2026

(54) Catheter Position Sensor with Electronic Position Adjustment

(71) Applicant: St. Jude Medical, Cardiology Division, Inc., St. Paul, MN (US)

(72) Inventors: William H. Peckels, Houlton, WI (US); Nicholas Sanger, Waterville, MN (US)

(73) Assignee: St. Jude Medical, Cardiology Division, Inc., St. Paul, MN (US)

(21) Appl. No.: 19/452,585

(22) Filed: Jan. 19, 2026

Related U.S. Application Data
(60) Provisional application No. 63/762,317, filed on Feb. 24, 2025.

(51) Int. Cl. A61M 25/01; A61M 25/00; A61M 25/06; A61M 39/00; A61M 39/06

(52) U.S. Cl. CPC A61M 25/0105; A61M 25/0043; A61M 25/0097; A61M 25/0662; A61M 39/06; A61M 2025/0004; A61M 2039/0009; A61M 2205/103; A61M 2205/3306; A61M 2205/502; A61M 2210/12

(57) ABSTRACT

A system for positioning a catheter within vasculature of a patient includes an introducer having a proximal hub configured to remain outside of the patient, an introducer catheter extending from the proximal hub, and a body forming a hub lumen. The system also includes the catheter, the catheter being sized and shaped to pass through the hub lumen and through the introducer catheter into the vasculature of the patient. The introducer includes a linear encoder sensor and the catheter includes a linear encoder scale so that, as the catheter passes through the introducer, the linear encoder sensor is configured to generate a signal indicative of an axial distance which the catheter has traveled relative to the introducer.

### CROSS-REFERENCE TO RELATED APPLICATIONS

This application claims priority to the filing date of U.S. Provisional Patent Application No. 63/762,317, filed Feb. 24, 2025, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

### SUMMARY OF THE DISCLOSURE

According to a first aspect of the disclosure, a system for positioning a catheter within vasculature of a patient includes an introducer having a proximal hub configured to remain outside of the patient, an introducer catheter extending from the proximal hub, and a body forming a hub lumen. The system also includes the catheter, the catheter being sized and shaped to pass through the hub lumen and through the introducer catheter into the vasculature of the patient. The introducer includes a linear encoder sensor and the catheter includes a linear encoder scale so that, as the catheter passes through the introducer, the linear encoder sensor is configured to generate a signal indicative of an axial distance which the catheter has traveled relative to the introducer. The linear encoder sensor may be a magnetic encoder sensor, and the linear scale may comprise a first group of magnets and a second group of magnets arranged on the catheter so that magnets of the first group alternate with magnets of the second group along a length of the catheter. In one example, (i) the magnets of the first group are ring magnets having an outer diameter and an inner diameter, a north pole of the magnets of the first group being positioned on the outer diameter and a south pole of the magnets of the first group being positioned on the inner diameter, and (ii) the magnets of the second group are ring magnets having an outer diameter and an inner diameter, a north pole of the magnets of the second group being positioned on the inner diameter and a south pole of the magnets of the second group being positioned on the outer diameter. In another example, the magnets of the first group are magnetic wires, and the magnets of the second group are magnetic wires, the magnetic wires of the first group and the magnetic wires of the second group being braided together with one or more non-magnetic wires to form a braided structure. The linear encoder sensor may be an optical encoder sensor, and the linear scale may comprise markings readable by the optical encoder sensor. The linear encoder sensor may be a camera, and the linear scale may comprise markings readable by the camera. The linear encoder sensor may be positioned on or in the body of the proximal hub in a position adjacent to the hub lumen. The proximal hub may include a hemostasis valve, the hemostasis valve being positioned distal to the linear encoder sensor and proximal to the introducer catheter.

The system may also include an attachment member, the attachment member including a body defining an attachment member lumen extending distally from a proximal end of the body, wherein the attachment member is configured to be coupled to the proximal hub to form a part of the introducer. The linear encoder sensor may be positioned on or in the body of the attachment member in a position adjacent to the attachment member lumen. The proximal hub may include a hemostasis valve, such that when the attachment member is coupled to the proximal hub, the hemostasis valve is positioned distal to the linear encoder sensor and proximal to the introducer catheter.

The linear encoder sensor may be positioned on a printed circuit board assembly (“PCBA”), a microcontroller being positioned on the PCBA so that the signal generated by the linear encoder sensor is capable of passing to the microcontroller. A signal amplifier may be positioned on the PCBA so that the signal generated by the linear encoder sensor is capable of passing to the signal amplifier before passing to the microcontroller. A noise filter may be positioned on the PCBA so that the signal generated by the linear encoder sensor is capable of passing to the noise filter before passing to the microcontroller. The microcontroller may be operably coupled to a display device configured to display information indicative of the axial distance which the catheter has traveled relative to the introducer. The introducer may include a motorized actuator configured to contact the catheter when the catheter is positioned within the introducer, the actuator configured to advance the catheter distally through the introducer. The actuator may also be configured to retract the catheter proximally through the introducer. The actuator may be a wheel actuator configured to rotate to advance the catheter distally. The introducer may include at least one button that, upon being depressed, sends a signal to the actuator to advance the catheter distally through the introducer. The actuator may be configured to receive a signal from the linear sensor encoder, and the actuator may be configured to advance the catheter distally based, at least in part, on the signal received from the linear encoder sensor. The introducer may include an idler and a biasing mechanism operably coupled to the idler, the biasing mechanism imparting force on the idler to push the idler toward the actuator. When the catheter is positioned through the introducer, the catheter may be in contact with both the actuator and the idler. The catheter may be a prosthetic heart valve delivery catheter having a balloon, and the system may further include a balloon-expandable prosthetic heart valve configured to be crimped onto the balloon for delivery while the balloon is in a deflated condition.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the handle of the delivery system shown in FIG. 4.

FIG. 6 is an enlarged view of a distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the inflation system of FIG. 8.

FIG. 10 is a perspective view of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIGS. 12A-12B are perspective and side views, respectively, of a hemostasis valve assembly according to an aspect of the disclosure.

FIGS. 12C-12D are cross-sections of the hemostasis valve assembly of FIGS. 12A-B in closed and open conditions, respectively.

FIG. 13A shows a sensor or encoder incorporated into the hemostasis valve assembly of FIG. 12C.

FIG. 13B shows a sensor or encoder incorporated into an attachment member, the attachment member being configured to couple to the hemostasis valve assembly of FIG. 12C.

FIG. 13C-1 is a schematic view of a catheter configured for use with the sensor or encoder of FIGS. 13A-B.

FIG. 13C-2 is a schematic view of another catheter configured for use with the sensor or encoder of FIGS. 13A-B.

FIG. 13D illustrates a magnetic encoder sensor or reader implemented on a printed circuit board assembly.

FIG. 14 is a flowchart showing example steps of an example method according to an aspect of the disclosure.

FIG. 15 shows various interrelated components of a prosthetic heart valve delivery system according to an aspect of the disclosure.

FIG. 16A is a schematic view of a proximal portion of an introducer according to an aspect of the disclosure.

FIG. 16B is a schematic view of a delivery catheter inserted into the proximal portion of the introducer of FIG. 16A.

FIG. 17 is a block diagram that illustrates an example of a computer system upon which an example may be implemented.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

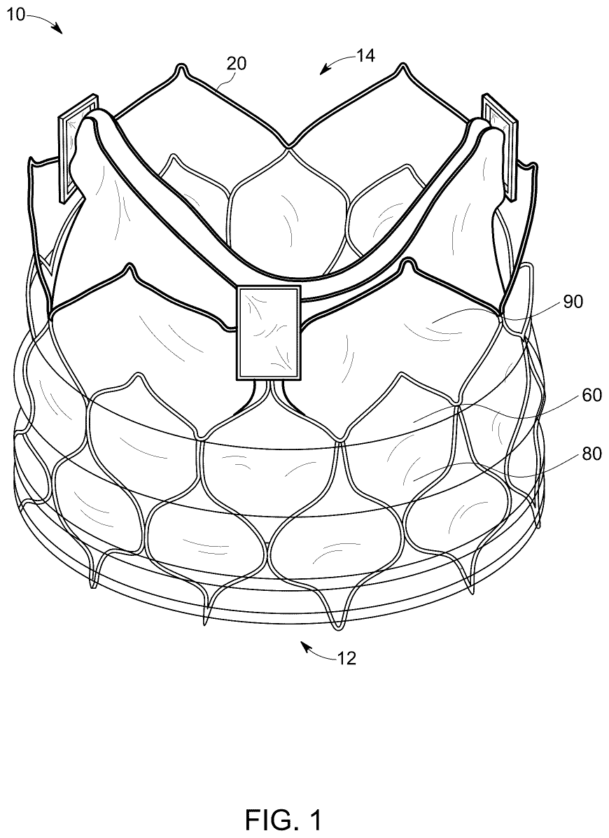

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

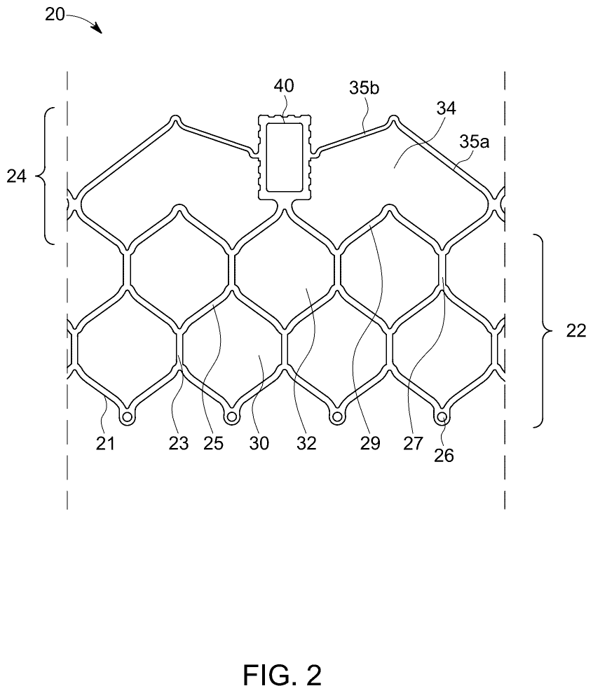

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g. fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. patent application Ser. No. 18/810,994, filed Aug. 21, 2024 and titled “TAVI Deployment Accuracy-Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g. struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

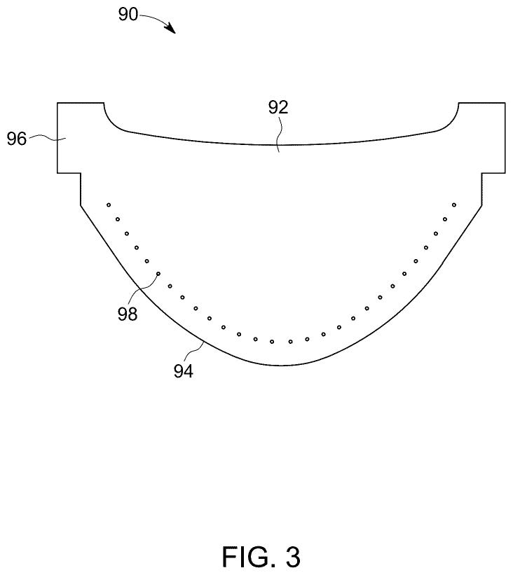

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other examples of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g. via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Laflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

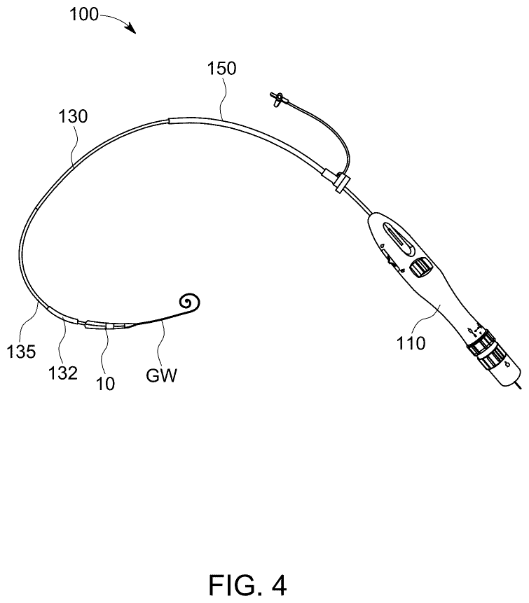

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

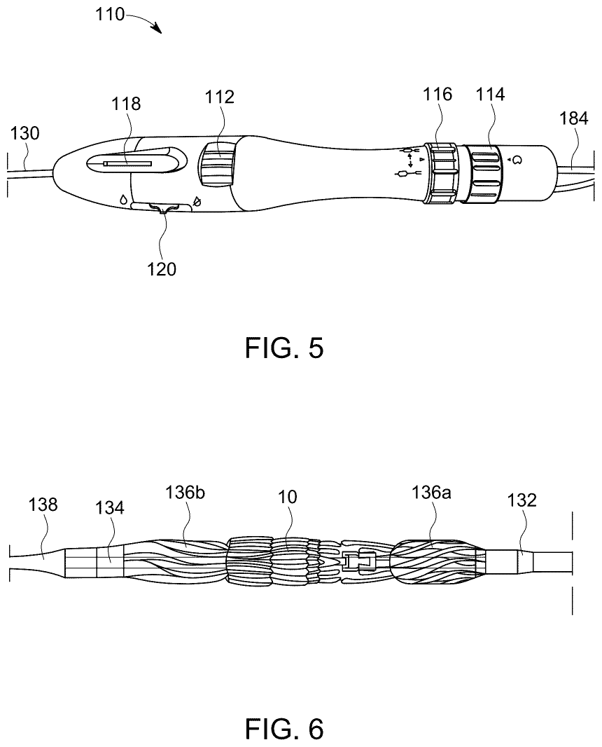

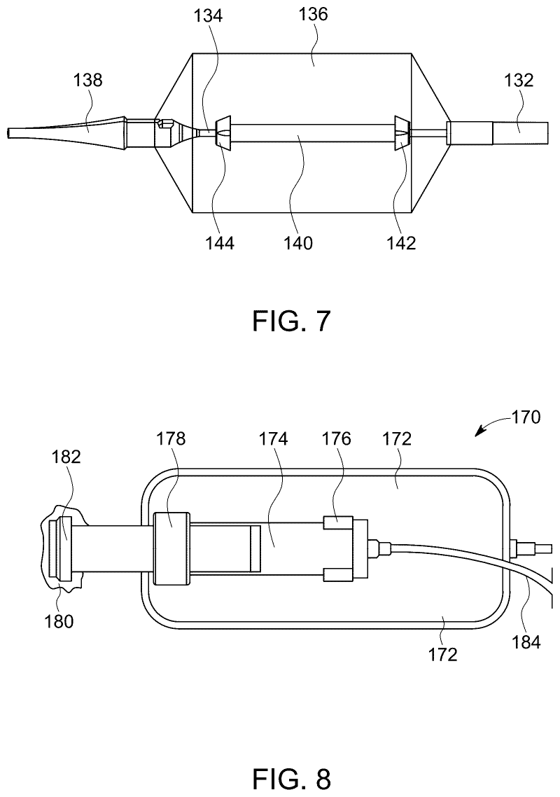

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135. In some examples, the handle may include a window 118 that allows viewing of an indicator that corresponds to the amount of catheter deflection. For example, a carrier to which the indicator is attached may be attached to the steering wire. In some examples, when there is minimum (or zero) tension on the steering wire, the indicator is at the far distal position within window 118, but as deflection is actuated, for example by drawing a carrier proximally (and tensioning the steering wire as the carrier draws proximally), the indicator will move proximally along window 118, giving the user a readily-apparent indication of the amount of deflection applied to the catheter at any given moment.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads (or another component, such as individual extensions, which may be cylindrical extensions that fit between internal threads of the actuator) of a carriage that is coupled to an inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it may have a small total range of motion, including for example between about 2 mm and about 15 mm of range of motion, including about 7.5 mm range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Patent Application Publication No. 2024/0148501, the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. In some embodiments, the balloon actuator 120 may take the form of a “momentary switch” in which pushing the balloon actuator 120 forward engages inflation, pulling the balloon actuator 120 proximally engages deflation, and releasing the balloon actuator 120 pauses inflation. This particular example of functionality may allow the physician to precisely control the amount of fluid dispensed while reducing the occurrence of over-or under-inflation, for example because the system automatically pauses inflation when the switch is released. The physical form factor of the balloon actuator 120 may be any suitable desired form factor, including for example a rocker switch, a push button, etc. In some embodiments a second balloon actuator or button may be provided, either on the balloon actuator 120 or elsewhere on the handle 110, with the second balloon actuator allowing for a change (e.g. increase or decrease) in the rate of inflation, for example to a pre-programmed faster or slower rate of inflation. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

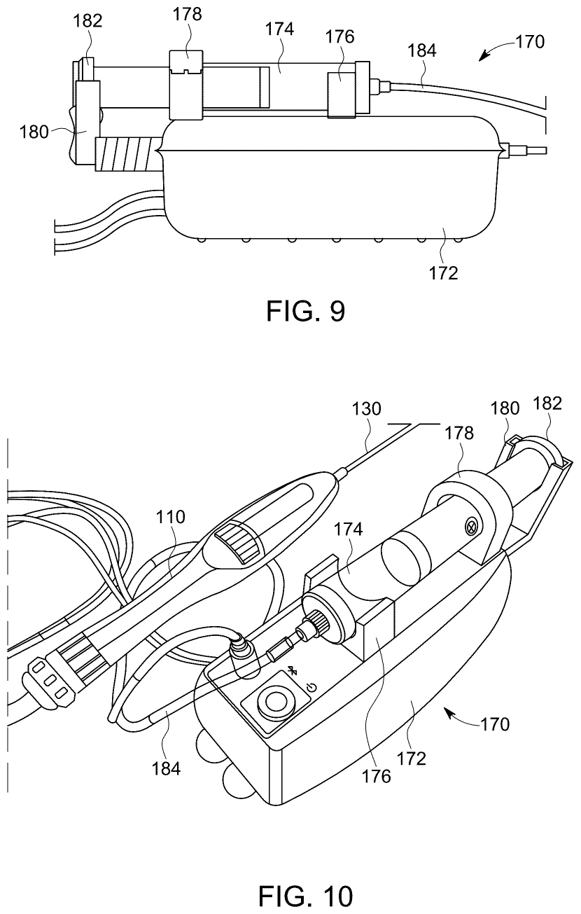

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in each of FIG. 8, FIG. 9, and FIG. 10, the distal end of syringe 174 may be coupled to tubing 184 that is in fluid communication with (i.e. fluidly coupled to) an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g. from AC mains or another component with which the cable is coupled) and/or transmission of data, information, control commands, etc. For example, one cable may couple the housing 172 to handle 110 so that controls on the handle 110 (e.g. balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. Patent Application Publication No. 2023/0372097, the disclosure of which is hereby incorporated by reference herein.

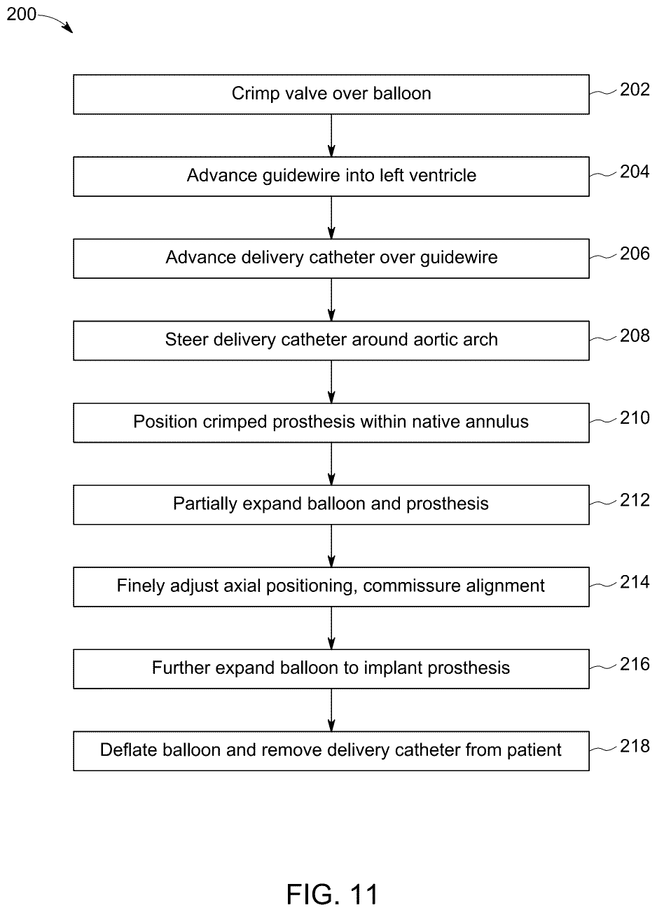

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g. by actuating axial alignment actuator 116) and/or the rotational orientation of the prosthetic heart valve 10 relative to the native aortic valve may be finely adjust (e.g. by actuating commissure alignment actuator 114). When the desired axial alignment is achieved and the desired rotational alignment (e.g. rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

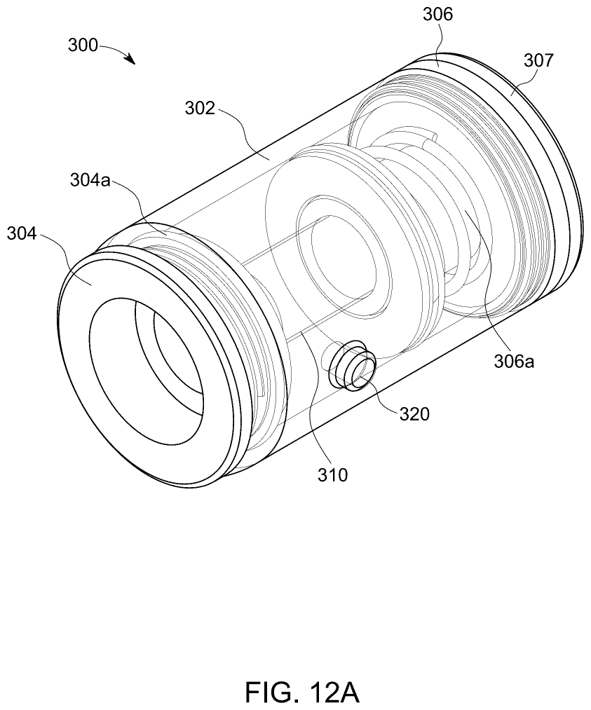

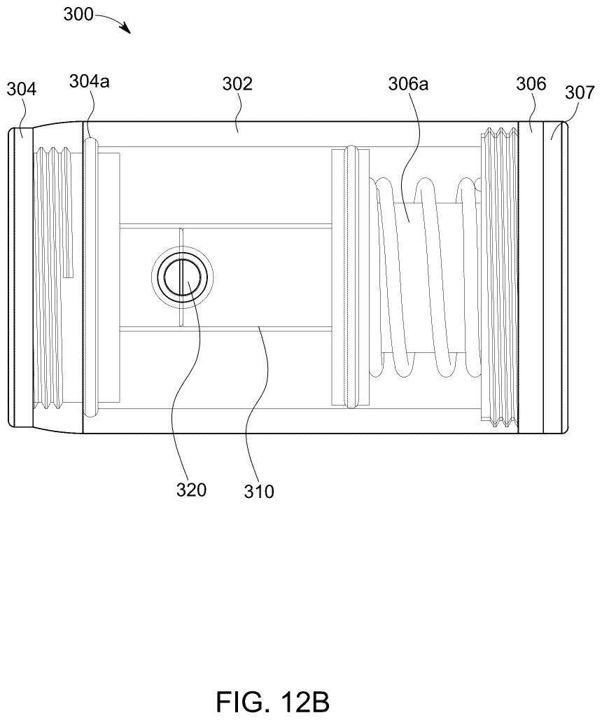

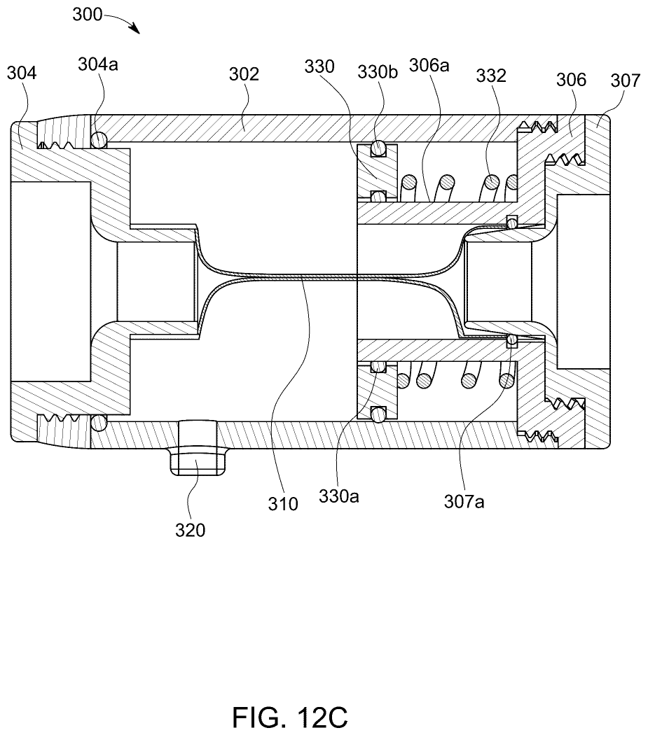

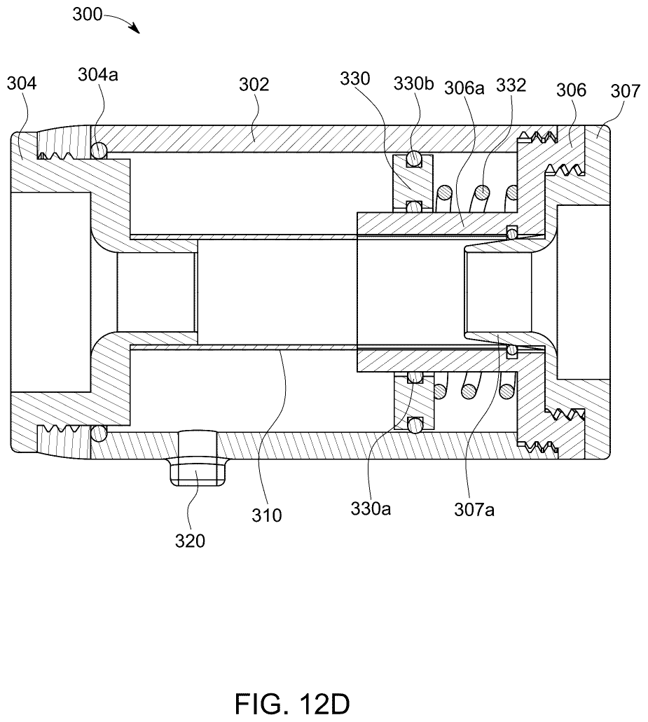

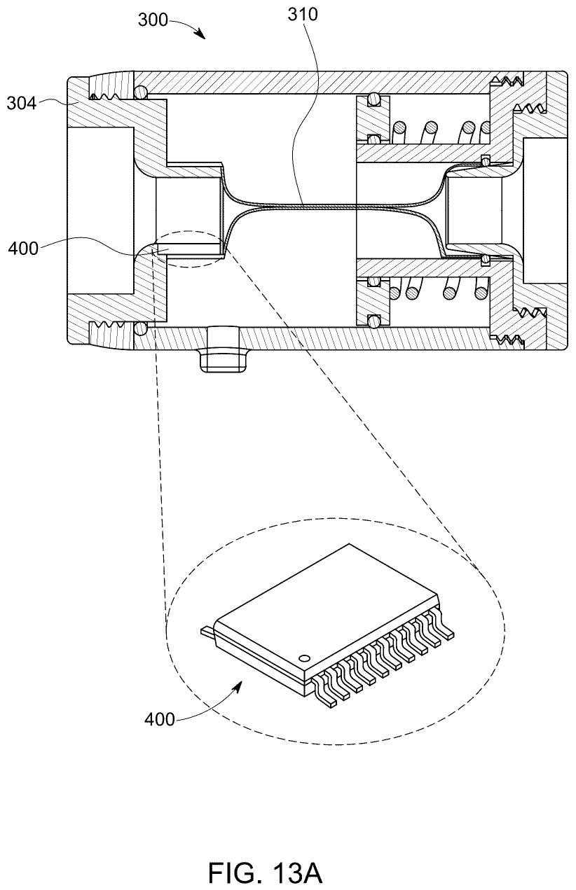

As noted above, delivery system 100 may be used with an introducer 150 that may have various different configurations. Introducers are used in many vascular procedures to assist in providing access of a treatment catheter into the patient's vasculature through the introducer. For example, introducer 150 may be inserted into a patient's femoral artery to provide access, with the remainder of the delivery system 100 (not including handle 110) being introduced into the patient's vasculature through the introducer 150. Typically, introducer 150 includes a hemostasis valve or seal at or near a proximal hub thereof to help ensure that blood does not travel from the patient's vasculature into and through the proximal end of the introducer 150 that remains outside the patient's body. In certain procedures, including transcatheter aortic valve replacement procedures, devices of significantly different sizes (e.g. diameters) need to be inserted into the patient through the introducer 150 at different times. For example, at one or more points in the procedure, the only device that may pass through the introducer 150 is a guidewire GW having a very small diameter. At other points, a device (such as delivery catheter 130 or an overlying component) having a diameter of up to about 36 French (12 mm) or 40 French (13.33 mm) more may pass through the introducer 150. In some examples, delivery catheter 130 has a size of about 14 French (4.66 mm). At other points in the procedure, no devices at all may pass through the introducer 150. It would be preferable for the hemostasis valve within introducer 150 to appropriately seal whether a large device, a small device, or no device passes through the introducer 150. It would also be preferable for the hemostasis valve provide an effective seal in all of these situations without the need for a user actively managing the seal (e.g. without having to actively manipulate or pressurize or depressurize the seal). Hemostasis valves that may provide this type of functionality are described in greater detail in U.S. Provisional Patent Application No. 63/711,182, titled “Hemostasis Valve for Vascular Device” and filed on Oct. 24, 2024, the disclosure of which is hereby incorporated by reference herein. One particular example of such a hemostasis valve is described in greater detail below in connection with FIGS. 12A-12D, although it should be understood that the details of this hemostasis valve are only exemplary.

FIGS. 12A-12B show a perspective and side views, respectively, of a hemostasis valve assembly 300 according to another aspect of the disclosure. Hemostasis valve assembly 300 may include a proximal fitting 304, and a first distal fitting 306. The proximal fitting 304 and first distal fitting 306 may each be connected to a housing or body 302. The housing 302 may be relatively rigid and may include an internal biasing mechanism to automatically or semi-automatically maintain a seal within the hemostasis valve assembly 300, whether a relatively large device, a relatively small device, or no device at all passes through the hemostasis valve assembly 300.

The hemostasis valve assembly 300 may be generally cylindrical (although variations from generally cylindrical shapes are possible). As best shown in FIGS. 12C-12D, the proximal fitting 304 may be a compression fitting that has an enlarged proximal head and an externally threaded shaft that may thread into a corresponding internally threaded proximal end of the housing 302. In this embodiment, a seal or gasket such as an O-ring 304a may be positioned between an external shoulder of the proximal fitting 304 and a corresponding internal shoulder of the proximal housing 302 so that, upon threading the proximal fitting 304 into the housing 302, the O-ring 304a is compressed and a fluid tight seal is created around the outer surface of the proximal fitting 304. In the illustrated embodiment, the proximal fitting 304 includes an internal lumen that may be generally cylindrical, with an outward flare or taper at the inlet or proximal end to help guide devices, such as catheters, into the proximal fitting 304.

The first distal fitting 306 may also be a compression fitting that has an enlarged distal head and an externally threaded shaft that may thread into a corresponding internally threaded distal end of the housing 302. In the illustrated embodiment, the distal fitting 306 includes an internal lumen that is generally cylindrical and which includes a smaller diameter proximal section, and a larger diameter distal section that is internally threaded. The internal threading of the larger diameter distal section allows for mating with external threading of a second distal fitting 307. The second distal fitting 307 may include a relatively large diameter distal lumen portion configured to mate with a catheter shaft for insertion into a patient, and a relatively small diameter proximal extension 307a that extends within a proximal extension 306a of the first distal fitting 306. A gasket such as an O-ring 307b may be positioned around the exterior of the proximal extension 307a and in contact with the interior surface of the proximal extension 706a to create a fluid-tight seal between the two proximal extensions.

A fluid bladder 310 (which may also be referred to as a membrane, sock, or extrusion) may be provided interior to the housing 302. It should be understood that, in the view of FIGS. 12A, 12B, and 12D, the fluid bladder 310 is in a fully or nearly fully open state, such that the interior lumen of proximal fitting 304 is in fluid communication with the interior lumen of distal fitting 306 through the interior of the open fluid bladder 310. On the other hand, in FIG. 12C, the fluid bladder 310 is in a fully or nearly fully closed state, such that the interior lumen of proximal fitting 304 is not in fluid communication with the interior lumen of distal fitting 306 through the interior of the open fluid bladder 310.

In some embodiments, the fluid bladder 310 is formed as an extrusion or laminate. The fluid bladder 310 is substantially non-permeable, such that fluid cannot flow across a wall of the fluid bladder 310, and is substantially non-elastic. Preferably, the material forming the fluid bladder 310 is lubricious so that devices passing from the proximal fitting 304 to the distal fitting 306 readily slide axially along the fluid bladder 310 without being caught on the material forming the fluid bladder 310. In some examples, the fluid bladder 310 may be formed from expanded polytetrafluoroethylene (“ePTFE”), a material with both high tensile strength and natural lubricity, although other materials may be suitable alternatives. The fluid bladder 310 in some examples may be generally cylindrical. A first or proximal end of the fluid bladder 310 may be coupled to a proximal portion of the housing 302, such as to the distal extension of the proximal fitting 304, for example via glue or other adhesives, the use of an overmolded component, or a clamp (e.g. hose-clamp) type mechanism. In other examples, the first or proximal end of the fluid bladder may be sandwiched between the proximal fitting 304 and O-ring 304a similar to other embodiments described herein. A second or distal end of the fluid bladder 310 may be coupled to the hemostasis valve assembly 300 by being sandwiched or pinched between the O-ring 307a and proximal extension of the first distal fitting 306 (or pinched between the O-ring 307a and the proximal extension of the second distal fitting 307. In other embodiments, the fluid bladder 310 may be otherwise sealingly secured against the housing 302 (and/or the fittings) so that, when fluid is pressurized between the outer surface of the fluid bladder 310 and the interior of the housing 302, that pressurized fluid will not escape the housing 302 from the proximal or distal ends of the housing 302.

Referring to FIG. 12C, the hemostatic valve may be created, in part, by injecting a non-compressible fluid, such as saline, through an inflation lumen 320. The inflation lumen 320 may be closed or locked, including for example via a one-way valve, so that the media cannot exit the inflation lumen 320. In the illustrated example, the fluid may fill the space interior of the housing 302, exterior to the fluid bladder 310, distal to the proximal fitting 304, and proximal to a piston 330 (described in greater detail immediately below), while forcing the bladder 310 to a closed or sealed position shown in FIG. 12C without significantly compressing the biasing member of spring 332 (also described in greater detail immediately below).

Referring to FIG. 12C, the hemostasis valve assembly 300 is shown after the housing 302 has been filled with a non-compressible fluid so as to force the fluid bladder 310 to close on itself to form a seal. Part of the volume in which the non-compressible fluid is maintained is bounded by a piston 330. In the illustrated example, the piston 330 is annular in shape with an inner opening through which the proximal extension 306a of the first distal fitting 306 extends. A gasket, such as O-ring 330a, may be positioned in contact with the interior surface of the inner opening of the piston 330 and the exterior surface of the proximal extension 306a. Similarly, a gasket, such as O-ring 330b, may be positioned in contact with the outer annular surface of the piston 330 and with an inner surface of the housing 302. With this configuration, as the piston 330 slides relative to the proximal extension 306a, the fluid within the housing 302 is not able to cross the piston 330 (e.g. between the piston 330 and the inner wall of the housing 302 or between the piston 330 and the outer surface of the proximal extension 306a).

Still referring to FIG. 12C, a biasing element such as a spring 332 may be positioned in contact with the surface of the piston 330 that does not confront the non-compressible fluid. In the illustrated examples, the biasing element takes the form of a spring 332 that is positioned around the proximal extension 306a, with ends of the spring 332 in contact with the piston 330 on one side and a face of the first distal fitting 306 on the other side. After the housing 302 is initially filled with non-compressible fluid to seal the fluid bladder 310, filling may be completed before the spring 332 begins to compress, or before it begins to significantly compress. In use, as a device (e.g. delivery catheter 130) passes through the proximal fitting 304, into the interior of the fluid bladder 310, and eventually into and through the distal fittings 306, 307, the fluid bladder 310 is forced to open which transfers force to the non-compressible fluid. Because the fluid is substantially non-compressible, instead of the fluid simply pressurizing, a volume of the fluid begins to displace the piston 330 in a direction that tends to compress the spring 332. FIG. 12D shows an example in which the fluid bladder 310 has opened, although it should be understood that the device passing through the hemostasis valve assembly 300 has been omitted for clarity of illustration. The displaced fluid within the housing 302 causes the piston 330 to slide and the spring 332 to compress, without losing any seal between the piston 330 and the housing 302 or proximal extension 306a. The compression of the spring 332 will tend to try to force the piston 330 back toward the fluid, which helps ensure that the fluid bladder 310 is held tightly over the device passing through the interior of the fluid bladder 310, thus maintaining a seal whether a small device, a large device, or no device passes through the hemostasis valve assembly 300.

Although not shown in detail in connection with FIGS. 12A-12D, it should be understood that a catheter may be coupled to the second distal fitting 307 in a use condition. In some examples, the second distal fitting 307 may include internal threading to connected to a catheter. In some examples, a catheter may be snap fit, friction fit, or otherwise connected to the second distal fitting 307. In some examples, a catheter may be formed integrally with second distal fitting 307. Hemostasis valve assembly 300 may provide for automatic and passive adjustment of the seal to accommodate devices of different diameters (or no device at all) passing through the hemostasis valve assembly 300 while the fluid bladder 310 maintains a good seal over the device, helping to ensure no fluid, including blood, leaks through the hemostasis valve assembly 300 during use.

Although one particular type of hemostasis valve assembly 300 is described, for example for use as a (or as part of a) proximal hub of an introducer (such as introducer 150), it should be understood that any specifics of valve assembly 300 and introducer 150 provided above are merely intended to provide one example of an introducer 150. The disclosure below focuses on mechanisms by which a catheter (e.g. similar to delivery catheter 130) passing through an introducer (e.g. similar to introducer 150) may be accurately tracked to assist with precise positioning of a catheter within a patient. Although these systems may be particularly useful for delivering prosthetic heart valves including those described above, it should be understood that the catheter tracking mechanisms described in greater detail below may be applicable to any system in which a catheter is advanced into a patient's vasculature via an introducer, whether or not a separate device is being delivered via the delivery catheter, and whether or not the introducer and/or catheter have similar features as introducer 150, delivery catheter 130, and/or hemostasis valve assembly 300.

In various procedures which include a catheter being advanced through the vasculature of a patient, it may be useful for the user to have precise positioning data regarding axial movement of the catheter through the patient's vasculature. In a typical procedure in which a catheter is advanced through a patient's vasculature, including for example a transcatheter prosthetic heart valve replacement procedure, catheters are manipulated by hand with little to no displacement feedback outside of general fluoroscopic imaging and user sensation with respect to the distance traveled axially. In some environments, including procedures in which a catheter is used within or near the patient's heart, including for example delivering a prosthetic implant into or near the heart or in electrophysiology-related procedures, precision of catheter placement becomes increasingly important. For example, it may be beneficial to have confidence of axial catheter positioning within accuracy of 1 mm increments of axial movement or smaller. At least some of the systems and/or methods described below may help to provide precise measurement feedback of insertion or withdrawal of the catheter relative to the patient's vasculature to the user based on the translation of the catheter via the physician's hand. In examples in which such systems and/or methods are used in the context of a transcatheter prosthetic heart valve delivery (or other transcatheter heart implant delivery, such as a clip for performing transcatheter edge-to-edge repair (“TEER”) of the mitral valve or tricuspid valve, or an occluder for closing an atrial septal defect (“ASD”) or a left atrial appendage (“LAA”), the precise positioning feedback may reduce the risk of deployment movements not captured by fluoroscopic imaging by increasing the movement resolution at the site of implantation. In some embodiments described below, integration of these systems and/or methods with ancillary catheter lab equipment (e.g. fluoroscopic imaging systems) may be included to provide clear visibility of distance traveled and/or other catheter positioning information to the user. As described in greater detail below, at least some examples of systems and/or methods described herein may provide quantitative feedback regarding catheter positioning to the user, which could be helpful in increasing the repeatability of a particular procedure between different users, for example by reducing or eliminating the feedback of user sensation associated with manual catheter movements.

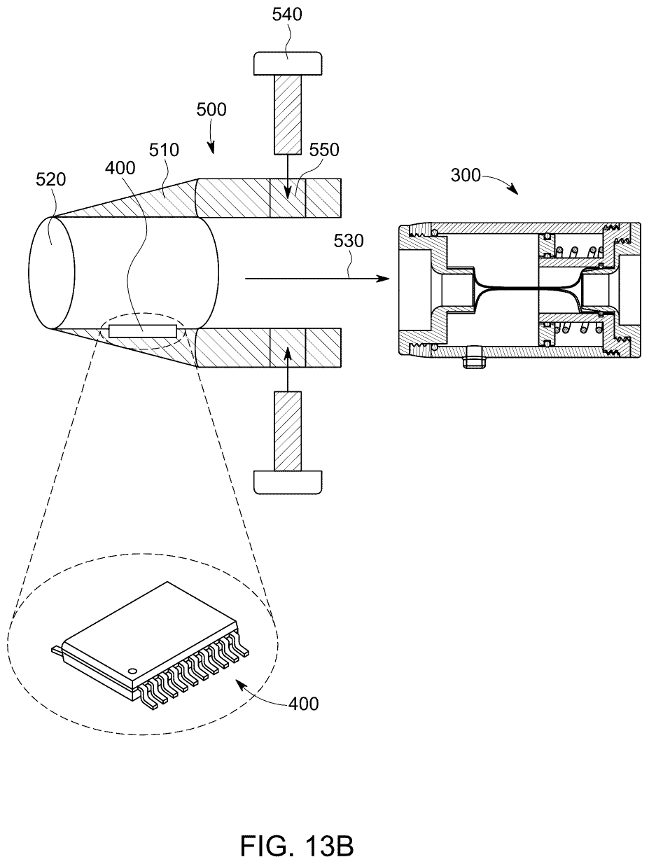

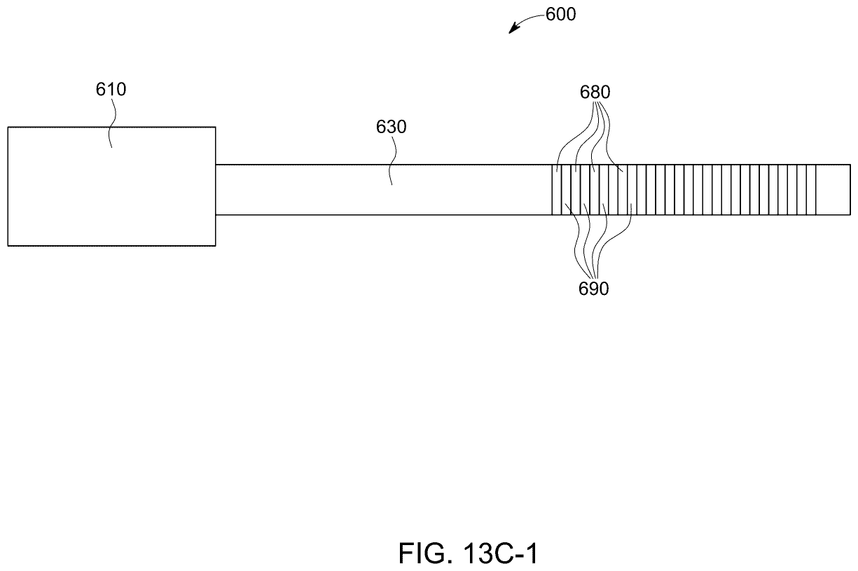

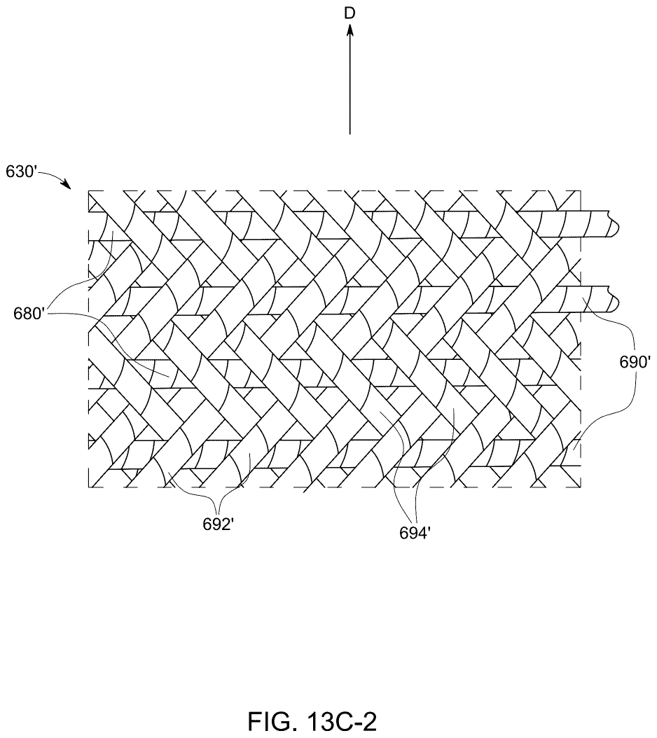

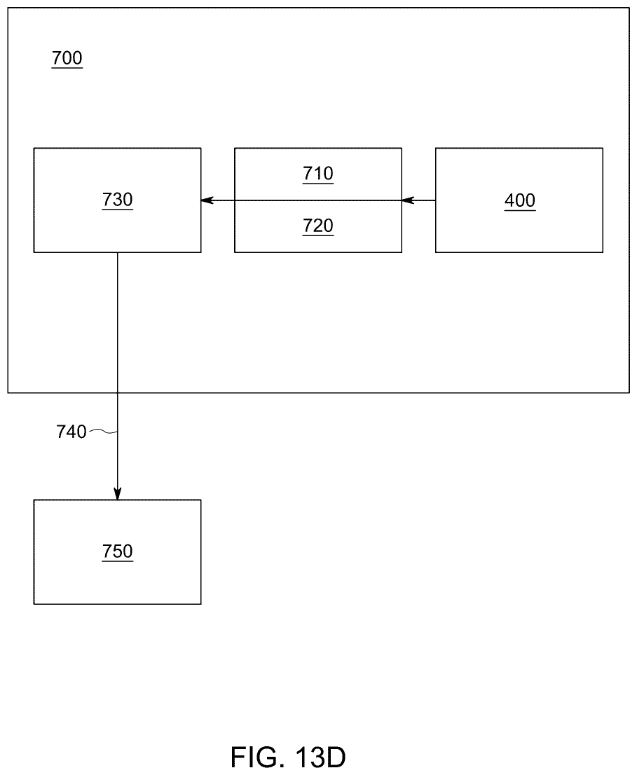

FIG. 13A shows one example of an implantation of a reader or sensor 400 of an encoder into hemostasis valve assembly 300. In the specific example of FIG. 13A, the sensor 400 is positioned on or in a portion of the proximal fitting 304 proximal to the fluid bladder 310, for example on a surface or embedded or positioned within a surface which is sized and positioned to receive another device (e.g. a catheter such as delivery catheter 130) therethrough. The sensor 400 and examples of use of the sensor 400 are described in greater detail below. Prior to that discussion, it should be understood that hemostasis valve assembly 300 is merely one example of a device into which sensor 400 may be incorporated. For example, other hemostasis valve assemblies of introducers (besides the specific implementation of hemostasis valve assembly 300), or proximal introducer hubs generally (whether or not such proximal hubs include a hemostasis valve) may include a sensor similar to sensor 400 without deviating from the scope of the invention. When included in other hemostasis valve assemblies and/or introducer hubs, the sensor 400 may be positioned in a location which will be in close proximity to movement of a catheter axially into or out of the introducer.

While FIG. 13A shows that the sensor 400 may be incorporated directly into an introducer hub and/or hemostasis valve assembly such as hemostasis valve assembly 300, in other examples the sensor 400 may be incorporated into an external or accessory device that is configured to couple to an introducer hub or hemostasis valve assembly that does not have such an integrated sensor 400. For example, FIG. 13B shows an example of an attachment member 500 (which may also be referred to as an external device or accessory device) which includes sensor 400. In the particular example of FIG. 13B, the attachment member 500 includes a body 510 defining a lumen extending from a proximal opening 520 (which may be sized and shaped to receive a leading end of a catheter therethrough) to a distal opening 530 which may be sized and shaped to be coupled to a proximal hub and/or hemostasis valve assembly of an introducer. In this particular example, hemostasis valve assembly 300 is shown as being a device over which the distal opening 530 may be positioned. The attachment member 500 may be configured to reversibly couple or lock onto the introducer hub and/or hemostasis valve assembly of the introducer, for example via a snap fit, friction fit, or mechanical interlock. In the illustrated example, the body 510 of the attachment member 500 includes one or more threaded openings 550 sized and shaped to receive one or more corresponding fasteners 540, which may be set screws. For example, after sliding the attachment member 500 over the proximal end of hemostasis valve assembly 300 via distal opening 530, the fastener(s) 540 may be screwed or otherwise advanced trough the openings 550 so as to press into engagement with the hemostasis valve assembly 300. In some examples, the hemostasis valve assembly and/or proximal hub of the introducer may include a complementary feature for receiving fasteners 540 (or any other locking mechanism). However, in other examples, the hemostasis valve assembly and/or proximal hub of the introducer does not include such complementary features. In other words, the attachment member 500 may be configured to couple to a large variety of sizes, shapes, brands, and types of introducers to provide functionality similar to that described below.

FIG. 13B illustrates that sensor 400 may be positioned on or in a portion of the body 510 proximal to where the proximal end of the hemostasis valve assembly 300 will be positioned when the attachment member 500 is attached to the hemostasis valve assembly 300. Thus, when the attachment member 500 is attached to the hemostasis valve assembly 300 (or any other hemostasis valve assembly and/or proximal hub of an introducer), the attachment member 500 defines a lumen that allows a catheter to pass into and through the attachment member 500 and into and through the introducer, with the catheter being moved axially in close proximity to the sensor 400.

FIG. 13C-1 is a schematic view of a catheter system 600 that may be configured for use with the sensor 400. Generally, catheter system may include a catheter shaft 630 and optionally a handle 610. Handle 610, if included, may be any suitable handle. For example, handle 610 may be similar or identical to handle 110. Catheter 630 may extend distally from the handle 610 and may be any suitable catheter for delivery into and/or through a patient's vasculature. For example, catheter 630 may be similar or identical to delivery catheter 130.

In one example in which sensor 400 is a magnetic sensor of a magnetic linear encoder, the catheter 630 may include a plurality of magnets disposed thereon. For example, part of, most of, or the entire length of the catheter 630 may include a first set of magnets 680, and a second set of magnets 690, so that magnets of the first set of magnets 680 alternate with magnets of the second set of magnets 690. In an example, each magnet 680 of the first set may be a ring-type magnet having the north pole on the outer diameter of the magnet, while each magnet 690 of the second set may be a ring-type magnet having the south pole on the outer diameter of the magnet (although the opposite configuration may also be suitable).

With the configuration described above, the catheter 630 may form, in effect, a magnetic scale which can be read by sensor 400 (whether sensor 400 is provided within a component of the introducer (e.g. introducer 150) itself, such as within a proximal hub and/or hemostasis valve assembly, or within a separate device (such as attachment member 500) attached to the introducer) as the catheter 630 is passed into or out of the introducer to create relative motion between the sensor 400 and the plurality of alternating-pole magnets 680, 690. In other words, the combination of the catheter 630 and the introducer may form a linear magnetic encoder (which may be configured as an absolute or incremental linear encoder) that is capable of discerning axial movement of the catheter 630 relative to the introducer, with relative axial motion as little as one micron (or even smaller increments) being discernable via the magnetic encoder.

FIG. 13C-2 illustrates another example of a catheter 630′ that may be part of a catheter system similar to catheter system 600. Catheter 630′ may include at least one braided layer forming a generally tubular shape configured to be inserted into a patient in an insertion direction D that aligns with the longitudinal axis of the shaft of the catheter 630′. Rather than including magnets such as ring-type magnets, the braided layer of catheter 630′ may include a plurality of functional braid wires, including for example a first group of magnetic braid wires 680′ having a first polarity, and second group of magnetic braid wires 690′ having a second polarity opposite the first polarity. In the illustrated example, the braid wires 680′ in the first group alternate with the braid wires 690′ in the second group, with each of the first and second braid wires 680′, 690′ extending in a direction generally perpendicular to the insertion direction D along the length of the shaft of the catheter 630′. The braided layer of catheter 630′ may include additional braid wires, which are preferably non-magnetic (e.g. plastic or fabric strands/wires), such as a third group of non-magnetic braid wires or strands 692′ and a fourth group of non-magnetic braid wires or strands 694′, which may both extend in directions different to the first and second groups of magnetic braid wires 680′, 690′. In some examples, the spacing between each braid wire 680′ of the first group and each adjacent braid wire 690′ of the second group is about the same, for example about 1 mm, about 2 mm, etc. This configuration may result in a generally similar functionality as catheter 630. For example, as the catheter 630′ is advanced through an introducer (or similar device) in the insertion direction D, or retracted from the introducer in a retraction direction that is opposite the insertion direction D, the braid wires 680′ of the first group and the braid wires 690′ of the second group will alternately pass by the magnetic sensor (e.g. sensor 400).

It should be understood that, although the magnetic encoder sensor 400 is shown as a generally isolated unit in FIGS. 13A-13B, in practice the sensor 400 may be integrated into a larger assembly that is coupled to the proximal hub of the introducer, to the hemostasis valve assembly, to the external attachment member, or to the particular desired introducer component. For example, FIG. 13D illustrates that the magnetic encoder sensor or reader 400 may be implemented on a printed circuit board assembly (“PCBA”) 700 that may include additional components. For example, the sensor 400 may be operably coupled other items, such as a signal amplifier 710 and/or a noise filter 720, which may also be positioned on the PCBA 700. If both a signal amplifier 710 and a noise filter 720 are provided, the signal from the magnetic sensor 400 may be first amplified by the signal amplifier 710 and then the amplified signal may be filtered by the noise filter 720, or the signal from the magnetic sensor 400 may be first filtered by the noise filter 720 and then the filtered signal may be amplified by the signal amplifier 710. After the signal from the magnetic encoder sensor 400 is passed through either or both of the signal amplifier 710 and the noise filter 720 (with the understanding that both components are optional), the signal may pass to a microcontroller unit (“MCU”) 730 which may be positioned on the PCBA 700. The MCU 730 may read the data received and transmit data to a display 750 (or to another system operably coupled to a display) to present relevant information relating to the axial positioning of the catheter 630 to the user. In some examples, the display 750 may be on the delivery system itself, such as on the handle 610. In some examples, the display 750 may be an external device, such as a tablet, a screen, or any other device capable of displaying data. In some examples, the data transmission between the MCU 730 and the display 750 may be via a wired connection 740, although in other examples it may be via wireless connection.

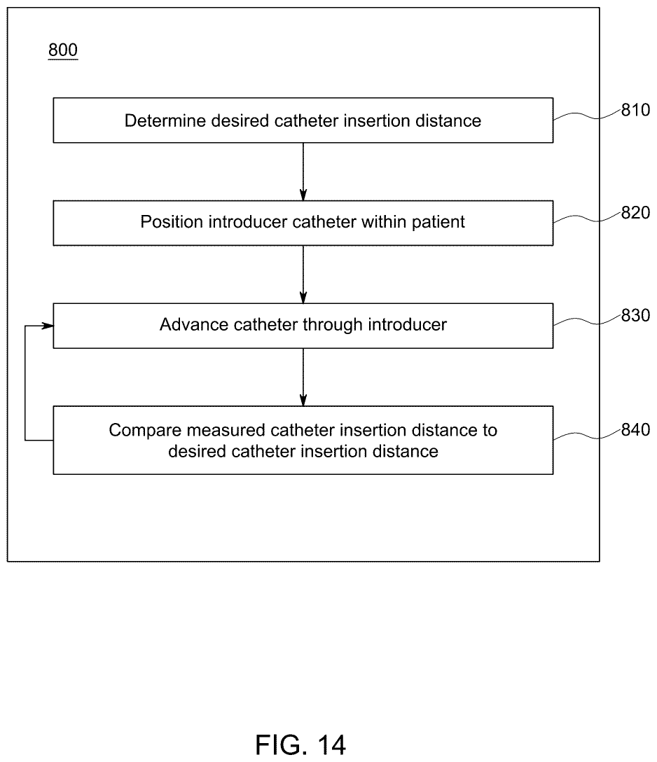

In some examples, the magnetic encoder sensor 400 and/or other related components (including those for example on PCBA 700) may be used in a procedure which involves manual axial advancement of the catheter 630 or 630′ through the vasculature. FIG. 14 is a flowchart showing example steps of an example method 800 according to an aspect of the disclosure. Method 800 may be implemented with any procedure in which a catheter is passed through the vasculature of a patient via an introducer and in which precision is desired regarding axial distance traveled for the catheter through the introducer and into the patient's vasculature. Method 800 may be implemented in catheter systems that do not result in a prosthesis being deployed into a patient (e.g. an ablation procedure within the heart) or in catheter systems that do result in a prosthesis being deployed (e.g. TAVR, TEER, LAA occlusion, etc.). In one specific example, method 800 may be performed as part of method 200, including for example as part of method steps 206, 208, and/or 210 of FIG. 11. For example, method 800 may include a step 810 in which a desired catheter insertion distance is determined. In some examples, step 810 may be performed as a single step using pre-procedural information. For example, medical imaging (e.g. CT, MRI, fluoroscopy, or any other suitable imaging modality) may be used to determine the total distance that the delivery catheter (e.g. delivery catheter 630 or 630′) should travel through the introducer and into the patient's vasculature to be positioned at an optimal position for treatment (e.g. deployment of a prosthesis). This determination may take into account, for example, the position of the magnetic encoder sensor 400 relative to the introducer (e.g. introduce 150), the expected position of the proximal hub of the introducer relative to the patient, and the total linear distance that must be traveled between the magnetic encoder sensor 400 and the target site so that optimal catheter positioning is achieved. Step 810 may be performed based solely on pre-procedural data, or otherwise may be performed based, at least in part, on procedural data such as the actual positioning of the introducer relative to the patient anatomy. Further, while step 810 may be performed a single time, it may also be performed multiple times during the procedure, for example to take into account new information and to update the optimal target distance for the delivery catheter 630 or 630′ to extend through the introducer. If step 810 is performed multiple times, the desired positioning value may be updated each time step 810 is performed. In some examples, the desired positioning value (which may be represented as a “total desired distance of travel” quantity) may be displayed on any display device accessible to the user. In some examples, the desired catheter insertion distance determined in step 810 may actually be an insertion distance relative to an anatomical landmark. For example, if it is desired to position the inflow edge of the prosthetic heart valve a particular distance (e.g. 1 mm, 3 mm, 5 mm etc.) below or sub-annular to the plane of the native heart valve annulus, the target distance may be 5 mm (if 5 mm sub-annular is the target), and the measured catheter insertion distance may be set or re-set to zero once the inflow edge of the prosthetic heart valve is positioned in alignment with the plane of the native heart valve annulus. This may provide the benefit of only needing a smaller target distance.

In step 820, the introducer (e.g. introducer 150) may be positioned within the patient to provide access to the patient's vasculature. If method 800 is being performed as part of method 200, this step may be performed prior to initially advancing the guidewire into the patient, or just prior to advancing the delivery catheter over the guidewire. As noted above, the magnetic encoder sensor 400 (and/or the PCBA 700 which includes the magnetic encoder sensor 400) may be positioned directly on a component of the introducer, such a portion of a hemostasis valve assembly 300 or another part of a proximal hub of the introducer, or otherwise may be positioned on an accessor member (e.g. attachment member 500) which may be separately coupled to the introducer, including either before or after the introducer is positioned within the patient.

Once the introducer is within the patient, the user may then advance the delivery catheter 630 or 630′ into the patient by advancing the delivery catheter 630 or 630′ through the introducer in step 830. If method 800 is being performed as part of method 200, step 830 may be performed after the prosthetic heart valve (e.g. prosthetic heart valve 10) has been crimped onto a balloon (e.g. balloon 136) of the delivery catheter (e.g. delivery catheter 130/630/630′). As described in greater detail below, as the alternating magnets 680, 690 lining the shaft of catheter 630 (or the alternating magnetic braid wires 680, 690′) pass in close proximity over the magnetic encoder reader 400, signals are generated (which signals may optionally be passed through the signal amplifier 710 and/or noise filter 720 before being passed to the MCU 730) indicative of axial distance traveled for the catheter 630 or 630′ relative to the introducer (e.g. relative to the position of the magnetic encoder reader 400 attached directly or indirectly to the introducer). Relevant distance-related information may be passed via a wired or wireless connection (e.g. connection 740) to a display 750, or to a system operatively coupled to a display 750, to be presented to the user in any useful format.

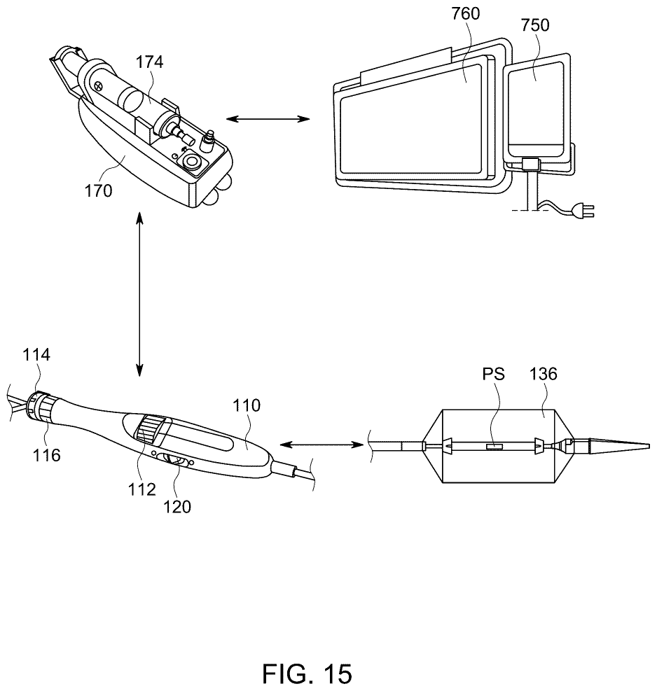

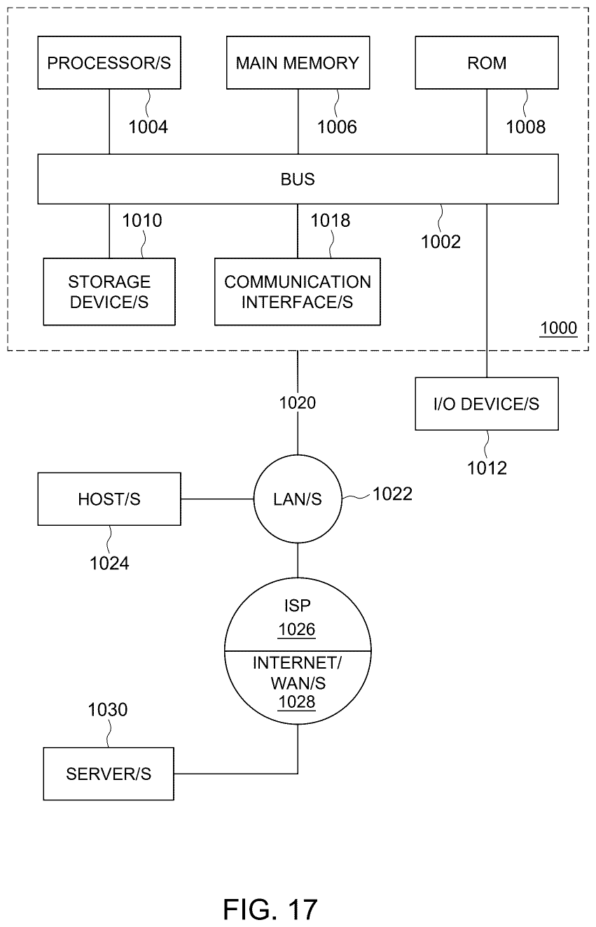

Referring briefly to FIG. 15, FIG. 15 shows a schematic view of balloon inflation system 170 with related components. In the illustrated example, the system includes a balloon inflation system 170 including syringe 174 mounted thereon. In some examples, the balloon inflation system 170 may be operatively coupled to a balloon catheter handle 110 (e.g., via a fluid line and cables that transmit power and/or data). As described above, the balloon catheter handle 110 may include balloon actuator 120, steering knob 112, commissure alignment actuator 114 and/or axial alignment actuator 116. The balloon catheter handle 110 may be operatively coupled to inflatable balloon 136. Although the delivery device 100 shown in FIG. 15 is not described above as having magnets, it should be understood that the groups of magnets 680, 690 on catheter 630 (or the groups of magnetic braid wires 680′, 690′ of catheter 630′) may be applied to the catheter 130 of delivery system 100. In some examples, a pressure sensor PS may be positioned anywhere within the path of the inflation lumen that extends between syringe 174 and the interior of the balloon 136. In the illustrated example, the pressure sensor PS is mounted to an internal shaft within the balloon 136, but it should be understood that this is only one exemplary position. For example, in some embodiments, pressure sensor PS may be provided within handle 110. The pressure sensor PS may be operatively coupled to the balloon inflation system 170 so that data (e.g., pressure readings) may be transmitted from the pressure sensor PS to the balloon inflation system 170. Balloon inflation system 170 may be operably coupled to balloon catheter handle 110 and may in some examples also be operably coupled to a computer 760 (which may have an integrated display) and/or to a mobile display 750, such as a tablet. Computer 760 and tablet/display 750 may in some examples individually, in combination, or along with other components, form a computer system (or a portion thereof) as described in connection with FIG. 17. The data connections between the balloon inflation system 170 and the balloon catheter handle 110 and/or computer 760 may be wired or wireless. In some examples, the computer 760 may receive real-time readings from pressure sensor PS (which may be relayed through inflation system 170), and those real-time readings may be graphically displayed on the computer 760 and/or on an associated tablet/display 750. Similarly, in some examples, data regarding the state of inflation of balloon 136, including for example volume of inflation media passed into the balloon 136, may be displayed on the computer 760 and/or on an associated tablet/display 750. For example, due at least in part to the motorized driving of syringe 174 by inflation system 170, the inflation system 170 may know at any point how much inflation media has been passed from the syringe 174 to the balloon 136 (or vice versa), which information may be transmitted to the computer 760 for display along with the pressure data.

Whether the magnetic encoder sensor 400 (and/or PCBA 700) is positioned directly on the introducer (not shown in FIG. 15) or on a component attached to the introducer, the wired connection 740 (or wireless connection) may be to the balloon inflation system 170, to the computer 760, to the tablet/display 750, or to a separate system/display. With this configuration, the information regarding distance traveled of the catheter 130/630/630′ relative to the introducer may be displayed in a convenient location, such as on tablet/display 750. In some examples, the desired catheter insertion distance from step 810 may be displayed (e.g. on tablet/display 750) as a static number, although that number may change to the extent that step 810 is performed multiple times to update the desired catheter insertion distance. In some, examples, the total distance of axial catheter distance traveled, for example as determined by the MCU 730 via the signals emanating from the magnetic encoder sensor 400, may also be displayed (e.g. on tablet/display 750). If the magnetic encoder is an absolute magnetic encoder, the total travel distance may be determined, for example, using absolute values received from the magnetic encoder sensor 400. If the magnetic encoder is an incremental magnetic encoder, the total travel distance may be determined, for example, by summing all increments of travel values received from the magnetic encoder sensor 400. The displayed measured catheter insertion distance may update at any desired interval (e.g. every 1 mm of travel, every 0.5 mm of travel, etc.) based on the resolution of the magnetic linear encoder (which, as described above, may be able to track relative movement in increments as small as 1 micron or even smaller) and/or based on increments determined to be clinically useful. For example, even if the magnetic linear encoder is capable of tracking distance in increments of 1 micron, it may be preferable to update the travel distance displayed in larger increments, such as 0.5 mm or 1.0 mm increments, for simplicity to the user. In some examples, the user may simply refer to the total travel distance provided on the display (e.g. tablet/display 750) and compare that to the desired total travel distance (which may or may not also be displayed) to determine how much farther the catheter 130/630/630′ needs to be advanced into the patient prior to achieving the optimal distance. This is an example of step 840 shown in FIG. 14. In some examples, additional information may be provided to assist the user in comparing the measured catheter insertion distance to the desired catheter insertion distance. For example, the MCU 730 and/or computer 760 may compare these values and display another value to the user, for example the remaining distance that the catheter 130/630/630′ needs to be advanced prior to achieving the previously-determined optimal insertion distance. In some examples, audible feedback, haptic feedback, or visual feedback (e.g. a color change in display values or additional information graphics) may be provided to indicate to the user that the catheter 130/630/630′ is close to achieving and/or has achieved the desired catheter insertion distance based on the measured catheter insertion distance. Steps 840 and 830 may be performed simultaneously and/or in cycles. If method 800 is being performed as part of method 200, the end result of method 800 may align with step 210 in which the crimped prosthetic heart valve 10 is at the optimal pre-deployment position relative to the native valve annulus based on the determined desired catheter insertion distance being equal (or about equal) to the measured catheter insertion distance. At this stage, deployment may continue, for example as described in connection with steps 212, 214, and 216. Further, although the method(s) has generally been described in connection to precisely tracking catheter advancement using quantitative information, any withdrawal of the catheter (including for example if the user overshoots the intended positioning and the catheter needs to be withdrawn to achieve optimal positioning, or simply to assist the user in understanding withdrawal of the catheter from the patient) may be similarly tracked.

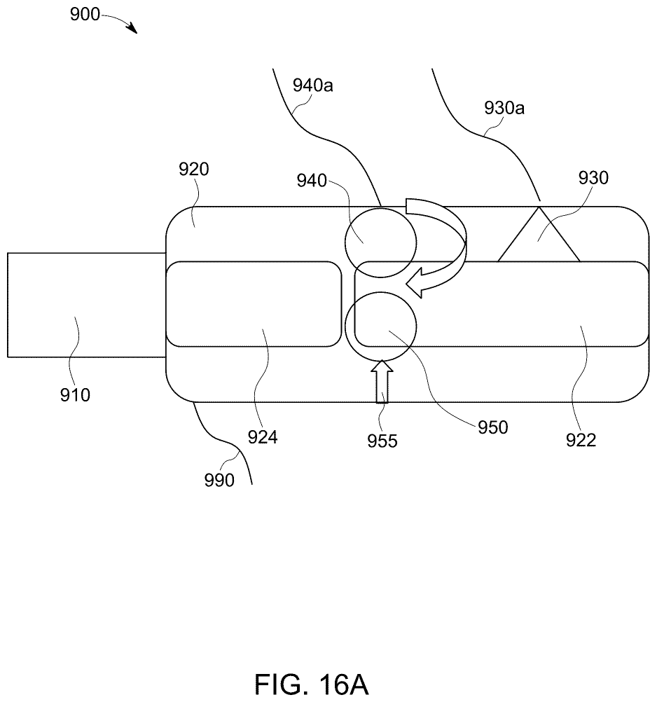

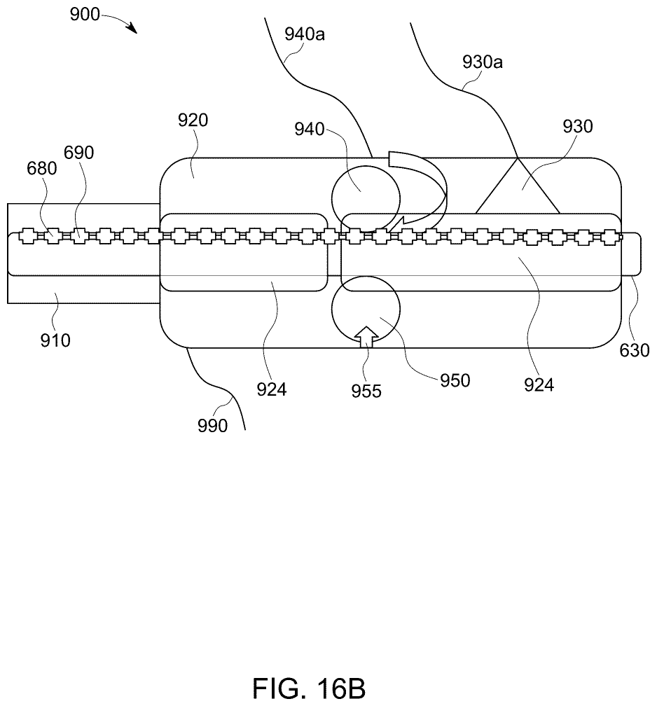

The above examples of using magnetic linear encoders to track catheter insertion distance generally relate to manual advancement (or withdrawal) of a catheter relative to an introducer, with quantitative distance information being provided to the user to help achieve highly precise catheter positioning. However, similar or identical systems and/or methods may be used with partially or fully automated advancement (or withdrawal) of a catheter relative to an introducer to achieve precise catheter positioning. For example, FIG. 16A is a schematic view of a proximal portion of an introducer 900 without any delivery catheter passing through the introducer 900. In the example of FIG. 16A, the introducer 900 includes an introducer sheath 910 extending distally from an introducer hub 920. The proximal introducer hub 920 may include a proximal opening or lumen 922 configured to receive a delivery catheter (e.g. delivery catheter 130, 630 or 630′) therethrough. The proximal hub 920 may also include a hemostasis valve 924, which may be any suitable hemostasis valve, including one having components similar or identical to those described in connection with hemostasis valve assembly 300. A flush line 990 may also be coupled to the proximal hub 920 to allow for flushing of interior components of the proximal hub 920, including for example the hemostasis valve 924, with any suitable fluid such as saline.

Similar or identical to as described above in connection with FIG. 13A, a magnetic encoder sensor 930 may be positioned in or on the proximal hub 920 in a location that is in close proximity to where the delivery catheter (e.g. catheter 130, 630 or 630′) will pass through. Encoder sensor 930 may be on a PCBA within the introducer hub 920, which PCBA may include other components including those shown and described in connection with FIG. 13D. FIG. 16A shows a representation of an encoder signal 930a which may be transmitted to other components of a PCBA within the introducer hub 920 on which the encoder sensor 930 is also positioned. In other examples, the encoder signal 930a may be transmitted to another device external to the introducer 900 for processing, including a system with similar features as shown and described in connection with FIG. 13D. The main difference of introducer 900 compared to earlier examples is that the introducer 900 may include a powered actuator 940 that is capable of automatically or semi-automatically positioning the catheter (e.g. catheter 130, 630 or 630′) relative to the introducer 900. In one example, the actuator 940 is a wheel or wheel-type mechanism that is operably coupled to a motor to rotate the actuator 940 about a central axis. In this example, at least a portion of the actuator wheel 940 extends into the proximal lumen 922 so that it is capable of contacting the external surface of a catheter (e.g. catheter 130, 630 or 630′) extending therethrough. FIG. 16A also shows a representation of an actuator signal 940a. In some examples, the magnetic encoder sensor 930 is positioned on a PCBA similar to PCBA 700, and the actuator signal 940a may be provided via a connection (e.g. wired or wireless) from the MCU 730. In some other examples, the actuator signal 940a may be provided from an external device to which the introducer 900 is coupled (e.g. wired or wirelessly). In some example, the introducer may include one or more internal power sources, such as rechargeable batteries, for powering the motor associated with actuator 940. In other examples, power may be provided via an external connection (e.g. via a connection of introducer 900 to AC mains, or via a wired connection to an external system).

Still referring to FIG. 16A, if the actuator 940 is a powered wheel-type actuator, the introducer hub 920 may also include an idler 950, which may also be a wheel type mechanism. In some examples, the idler 950 may extend at least partially into the lumen 922 of the proximal hub 920. In some examples, a biasing mechanism such as a spring 955 may tend to push the idler 950 toward the actuator 940. As described in greater detail below, the idler 950 may help to ensure good contact between the actuator 940 and the catheter (e.g. catheter 130, 630 or 630′) to help ensure good transmission of rotational movement of the actuator 940 to linear movement of the catheter. In some examples, the idler 950 may be passive without any active rotation. In other examples, the idler 950 may be powered and configured to rotate synchronously (but in the opposite direction) with actuator 940.

FIG. 16B shows the introducer 900 of FIG. 16A after delivery catheter 630 has been inserted therethrough (although catheter 630′ could be used in the same fashion). As shown in FIG. 16B, catheter 630 may include the same configuration of alternating magnets 680, 690 as described in connection with FIG. 13C-1 (or the same configuration of alternating magnetic braid strands 680′, 690′ of catheter 630′ as described in connection with FIG. 13C-2). As shown in FIG. 16B, the catheter 630 contacts the actuator 940 and forces the idler 950 to move away from the actuator 940, while the spring 955 compresses and imparts a larger force on the idler 950 to push the idler 950 against the other side of the catheter 630. In other words, the catheter 630 remains frictionally engaged with both the actuator 940 and idler 950, at least in part due to the force applied by spring 955. It should be understood that, in other embodiments, magnets 680, 690 shown in FIG. 16B could be replaced with other features to be sensed, for example alternating stripes of color or color patterns, with the encoder sensor 930 in such embodiments being an optical-type encoder sensor instead of a magnetic-type encoder sensor.

In an exemplary use, as the catheter 630 or 630′ translates relative to the magnetic encoder sensor 930, the encoder signal 930a is processed to track such movement and measure catheter insertion distance. In some examples, the desired catheter insertion distance (e.g. as determined in step 810) may be stored on the PCBA or on another computer system operably connected to the actuator 940. The actuator signal 940a may cause the actuator 940 to actuator (e.g. to rotate in the case that the actuator 940 is a wheel) to advance the catheter distally (to the left in the view of FIG. 16B) or to withdraw the catheter proximally (to the right in the view of FIG. 16B). Any such movement is tracked by the encoder sensor 930 (and related components), and the total travel distance may be compared to the desired travel distance (similar to step 840), with the actuator signal 940a being adjusted accordingly. For example, the actuator signal 940a may instruct the actuator 940 to continue rotating at the same rate, to increase the rate of rotation, to decrease the rate of rotation, to stop rotating, or to reverse the direction of rotation, depending on the difference between the measured travel of distance compared to the desired travel of distance. Stated in another way, method 800 may apply similarly to the embodiment of FIGS. 16A-B, with step 830 being automatic instead of manual. However, it should be understood that the catheter 630 or 630′ could still be manually advanced or retracted, with such movement still being captured via the magnetic encoder reader 930 (and related components). Further, although the actuator 940 is generally described above as being automatically actuated based on the actuator signal 940a, in other examples controls may be provided on (or operably connected to) the introducer 900. For example, a button may be provided to either allow for the actuator 940 to be actuated according to the actuator signal 940a (e.g. similar to a dead man's switch), or to stop actuation (e.g. a button may be pressed to pause the otherwise automatic actuation). In other examples, the actuator signal 940a may be based on an input signal from the user, instead of (or in addition to) information comparing the measured travel distance to the desired travel distance. For example, the introducer 900 (or another device operably coupled to the introducer 900) may include an advancement button and a retraction button that, upon being pressed, send a signal to the actuator 940 to rotate in one direction to advance the catheter 630 or 630′, or the opposite direction to retract the catheter 630 or 630′. With this embodiment, it may be left up to the user to compare the measured distance of travel and the desired distance of travel (e.g. similar or identical to step 840) and to use the buttons to actuator the actuator 940 accordingly. In still further examples, semi-automated controls may be provided where the user is largely in control of pressing one button to advance the catheter 630 or 630′ or another button to retract the catheter 630 or 630′, but upon the measured catheter travel distance reaching, or nearly reaching, the desired catheter travel distance, the actuator signal 940a may be automatically modified, for example to slow and then stop the actuation of actuator 940 to help prevent misplacement of the catheter 630 or 630′. However, in such examples, the user may be able to override such semi-automated controls. For example, even if the actuator signal 940a is modified to stop travel of the catheter once the measured travel distance matches the desired travel distance, the user may be able to release the button for catheter advancement, and then again depress the button for catheter advancement, to override the safety controls and ensure the catheter moves as the user desires.

Although the systems and methods described above are generally disclosed in connection with the use of magnetic encoder systems that include alternating magnets on the catheter with a reader on (or operably coupled to) the introducer, it should be understood that other types of linear encoders may be used in place of the magnetic linear encoder(s) described herein. For example, instead of using alternating magnets (or alternating magnetic braid wires) on the delivery catheter, marks may be placed on the catheter that are ready optically, including for example via a light beam or laser passing through the mark and being read by an optical sensor (which would be in place of the magnetic sensor 400 or 930). Some examples of markings including, for example, creating a “zebra-stripe” extrusion pattern when forming the catheter shaft 630 or 630′, which could include sequences of alternating colors or color patterns. Other types of linear encoders may be used in place of either a magnetic linear encoder or an optical linear encoder. Still in other examples, instead of a magnetic sensor 400 or 930, a camera may be used which creates a picture of the catheter, and the catheter may include any readable mark that is recognizable by the camera to determine either information of relative movement or absolute positioning of the catheter relative to the introducer. Thus, it should be understood that the specific embodiment(s) of the magnetic linear encoder sensor may be more generally referred to as a linear encoder sensor or reader, and the specific embodiment(s) of magnets with alternating poles may be more generally referred to as a linear encoder scale. Generally, the linear encoder sensor may encompass magnetic and other (e.g. optical) linear encoder sensors, and the linear encoder scale may encompass magnetic and other (e.g. optical) linear encoder scales that are capable of being read or sensed by the reader or sensor.

Although the catheter position tracking systems described above or generally described in the context of targeting a single desired position for a prosthetic heart valve delivery, it should be understood that the catheter position tracking systems may be used in other types of catheter systems, including ones that have multiple distance targets. For example, if the catheter position tracking system is being used for an ablation catheter, there may be multiple desired target distances corresponding to individual ablation sites. In such examples, a plurality of target positions (or target distances-of-travel) may be set, and the system may guide the user to the first target position at which a first ablation may be performed, and then the system may update the distance-of-travel target to the next sequential target, helping the user guide the catheter to the second target to perform a second ablation, and so forth.

Further, although embodiments are described herein in which a catheter includes a scale (e.g. linear encoder scale) while an introducer (or a portion thereof) includes a sensor (e.g. a linear encoder sensor) to read the scale, in some examples, the scale may instead be provided on the introducer (or a portion thereof) and the sensor (or multiple sensors) may be provided on the catheter so that the catheter sensor(s) read the scale on the introducer during relative axial motion therebetween. For example, a plurality of sensors on the catheter may iteratively read the scale on the introducer as the catheter passes through the introducer, and based on which sensor is reading the scale at a particular time (the scale in this instance may be a single data point or multiple data points), the system may determine the axial position of the catheter relative to the introducer.

Some of the techniques described herein, including computer-related and processor-related techniques relating to operation of the inflation system 170, magnetic encoder sensor 400, PCBA 700, magnetic encoder sensor 930, computer 760, and/or tablet/display 750, may be implemented in some examples at least in part by one or more special-purpose computing devices. The disclosure described below may apply to each of the inflation system 170, magnetic encoder sensor 400, PCBA 700, magnetic encoder sensor 930, computer 760, and/or tablet/display 750 as either individual components or components working in unison. The special-purpose computing devices may be hard-wired to perform one or more techniques described herein, including combinations thereof. Alternatively and/or in addition, the one or more special-purpose computing devices may include digital electronic devices such as one or more application-specific integrated circuits (ASICs) or field-programmable gate arrays (FPGAs) that are persistently programmed to perform the techniques. Alternatively and/or in addition, the one or more special-purpose computing devices may include one or more general-purpose hardware processors programmed to perform the techniques described herein pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices, and/or any other device that incorporates hard-wired or program logic to implement the techniques.

FIG. 17 is a block diagram that illustrates a computer system upon which an example may be implemented. The computer system 1000 may include a bus 1002 or other communication mechanism for communicating information, and one or more hardware processors 1004 coupled with bus 1002 for processing information, such as computer instructions and data. The processor/s 1004 may include one or more general-purpose microprocessors, graphical processing units (GPUs), coprocessors, central processing units (CPUs), and/or other hardware processing units.

The computer system 1000 may also include one or more units of main memory 1006 coupled to the bus 1002, such as random-access memory (RAM) or other dynamic storage, for storing information and instructions to be executed by the processor/s 1004. Main memory 1006 may also be used for storing temporary variables or other intermediate information during execution of instructions to be executed by the processor/s 1004. Such instructions, when stored in non-transitory storage media accessible to the processor/s 1004, may turn the computer system 1000 into a special-purpose machine that is customized to perform the operations specified in the instructions. In some embodiments, main memory 1006 may include dynamic random-access memory (DRAM) (including but not limited to double data rate synchronous dynamic random-access memory (DDR SDRAM), thyristor random-access memory (T-RAM), zero-capacitor (Z-RAM™)) and/or non-volatile random-access memory (NVRAM).

The computer system 1000 may further include one or more units of read-only memory (ROM) 1008 or other static storage coupled to the bus 1002 for storing information and instructions for the processor/s 1004 that are either always static or static in normal operation but reprogrammable. For example, the ROM 1008 may store firmware for the computer system 1000. The ROM 1008 may include mask ROM (MROM) or other hard-wired ROM storing purely static information, programmable read-only memory (PROM), erasable programmable read-only memory (EPROM), electrically-erasable programmable read-only memory (EEPROM), another hardware memory chip or cartridge, or any other read-only memory unit.

One or more storage devices 1010, such as a magnetic disk or optical disk, is provided and coupled to the bus 1002 for storing information and/or instructions. The storage device/s 1010 may include non-volatile storage media such as, for example, read-only memory, optical disks (such as but not limited to compact discs (CDs), digital video discs (DVDs), Blu-ray discs (BDs)), magnetic disks, other magnetic media such as floppy disks and magnetic tape, solid-state drives, flash memory, optical disks, one or more forms of non-volatile random-access memory (NVRAM), and/or other non-volatile storage media. The computer system 1000 may be coupled via the bus 1002 to one or more input/output (I/O) devices 1012. For example, the I/O device/s 1012 may include one or more displays for displaying information to a computer user, such as a cathode ray tube (CRT) display, a Liquid Crystal Display (LCD) display, a Light-Emitting Diode (LED) display, a projector, and/or any other type of display.

The I/O device/s 1012 may also include one or more input devices, such as an alphanumeric keyboard and/or any other keypad device. In some examples, the balloon actuators described herein may be an input device. The one or more input devices may also include one or more cursor control devices, such as a mouse, a trackball, a touch input device, or cursor direction keys for communicating direction information and command selections to the processor 1004 and for controlling cursor movement on another I/O device (e.g. a display). A cursor control device typically has degrees of freedom in two or more axes, (e.g. a first axis x, a second axis y, and optionally one or more additional axes z), that allows the device to specify positions in a plane. In some embodiments, the one or more I/O device/s 1012 may include a device with combined I/O functionality, such as a touch-enabled display.

Other I/O device/s 1012 may include a fingerprint reader, a scanner, an infrared (IR) device, an imaging device such as a camera or video recording device, a microphone, a speaker, an ambient light sensor, a pressure sensor, an accelerometer, a gyroscope, a magnetometer, another motion sensor, or any other device that can communicate signals, commands, and/or other information with the processor/s 1004 over the bus 1002.

The computer system 1000 may implement the techniques described herein using customized hard-wired logic, one or more ASICs or FPGAs, firmware, and/or program logic which, in combination with the computer system causes or programs, causes computer system 1000 to be a special-purpose machine. In some examples, the techniques herein are performed by the computer system 1000 in response to the processor/s 1004 executing one or more sequences of one or more instructions contained in main memory 1006. Such instructions may be read into main memory 1006 from another storage medium, such as the one or more storage device/s 1010. Execution of the sequences of instructions contained in main memory 1006 causes the processor/s 1004 to perform the process steps described herein. In alternative embodiments, hard-wired circuitry may be used in place of or in combination with software instructions.

The computer system 1000 may also include one or more communication interfaces 1018 coupled to the bus 1002. The communication interface/s 1018 provide two-way data communication over one or more physical or wireless network links 1020 that are connected to a local network 1022 and/or a wide area network (WAN), such as the Internet. For example, the communication interface/s 1018 may include an integrated services digital network (ISDN) card, cable modem, satellite modem, or a modem to provide a data communication connection to a corresponding type of telephone line. Alternatively and/or in addition, the communication interface/s 1018 may include one or more of: a local area network (LAN) device that provides a data communication connection to a compatible local network 1022; a wireless local area network (WLAN) device that sends and receives wireless signals (such as electrical signals, electromagnetic signals, optical signals or other wireless signals representing various types of information) to a compatible LAN; a wireless wide area network (WWAN) device that sends and receives such signals over a cellular network; and other networking devices that establish a communication channel between the computer system 1000 and one or more LANs 1022 and/or WANs. The network link/s 1020 typically provides data communication through one or more networks to other data devices. For example, the network link/s 1020 may provide a connection through one or more local area networks 1022 (LANs) to one or more host computers 1024 or to data equipment operated by an Internet Service Provider (ISP) 1026. The ISP 1026 provides connectivity to one or more wide area networks 1028, such as the Internet. The LAN/s 1022 and WAN/s 1028 use electrical, electromagnetic, or optical signals that carry digital data streams. The signals through the various networks and the signals on the network link/s 1020 and through the communication interface/s 1018 are example forms of transmission media, or transitory media.

The term “storage media” as used herein refers to any non-transitory media that stores data and/or instructions that cause a machine to operate in a specific fashion. Such storage media may include volatile and/or non-volatile media. Storage media is distinct from but may be used in conjunction with transmission media. Transmission media participates in transferring information between storage media. For example, transmission media includes coaxial cables, copper wire and fiber optics, including traces and/or other physical electrically conductive components that comprise the bus 1002. Transmission media can also take the form of acoustic or light waves, such as those generated during radio-wave and infra-red data communications.

Various forms of media may be involved in carrying one or more sequences of one or more instructions to the processor 1004 for execution. For example, the instructions may initially be carried on a magnetic disk or solid-state drive of a remote computer. The remote computer can load the instructions into its main memory 1006 and send the instructions over a telecommunications line using a modem. A modem local to the computer system 1000 can receive the data on the telephone line and use an infra-red transmitter to convert the data to an infra-red signal. An infra-red detector can receive the data carried in the infra-red signal and appropriate circuitry can place the data on the bus 1002. The bus 1002 carries the data to main memory 1006, from which the processor 1004 retrieves and executes the instructions. The instructions received by main memory 1006 may optionally be stored on the storage device 1010 either before or after execution by the processor 1004.

The computer system 1000 can send messages and receive data, including program code, through the network(s), the network link 1020, and the communication interface/s 1018. In the Internet example, one or more servers 1030 may transmit signals corresponding to data or instructions requested for an application program executed by the computer system 1000 through the Internet 1028, ISP 1026, local network 1022 and a communication interface 1018. The received signals may include instructions and/or information for execution and/or processing by the processor/s 1004. The processor/s 1004 may execute and/or process the instructions and/or information upon receiving the signals by accessing main memory 1006, or at a later time by storing them and then accessing them from the storage device/s 1010.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for positioning a catheter within vasculature of a patient, the system comprising:
an introducer having a proximal hub configured to remain outside of the patient, an introducer catheter extending from the proximal hub, and a body forming a hub lumen; and
the catheter, the catheter being sized and shaped to pass through the hub lumen and through the introducer catheter into the vasculature of the patient;
wherein the introducer includes a linear encoder sensor and the catheter includes a linear encoder scale so that, as the catheter passes through the introducer, the linear encoder sensor is configured to generate a signal indicative of an axial distance which the catheter has traveled relative to the introducer.

2. The system of claim 1, wherein the linear encoder sensor is a magnetic encoder sensor, and the linear scale comprises a first group of magnets and a second group of magnets arranged on the catheter so that magnets of the first group alternate with magnets of the second group along a length of the catheter.

3. The system of claim 2, wherein (i) the magnets of the first group are ring magnets having an outer diameter and an inner diameter, a north pole of the magnets of the first group being positioned on the outer diameter and a south pole of the magnets of the first group being positioned on the inner diameter, and (ii) the magnets of the second group are ring magnets having an outer diameter and an inner diameter, a north pole of the magnets of the second group being positioned on the inner diameter and a south pole of the magnets of the second group being positioned on the outer diameter.

4. The system of claim 2, wherein the magnets of the first group are magnetic wires, and the magnets of the second group are magnetic wires, the magnetic wires of the first group and the magnetic wires of the second group being braided together with one or more non-magnetic wires to form a braided structure.

5. The system of claim 1, wherein the linear encoder sensor is an optical encoder sensor, and the linear scale comprises markings readable by the optical encoder sensor.

6. The system of claim 1, wherein the linear encoder sensor is a camera, and the linear scale comprises markings readable by the camera.

7. The system of claim 1, wherein the linear encoder sensor is positioned on or in the body of the proximal hub in a position adjacent to the hub lumen.

8. The system of claim 7, wherein the proximal hub includes a hemostasis valve, the hemostasis valve being positioned distal to the linear encoder sensor and proximal to the introducer catheter.

9. The system of claim 1, further comprising:
an attachment member, the attachment member including a body defining an attachment member lumen extending distally from a proximal end of the body, wherein the attachment member is configured to be coupled to the proximal hub to form a part of the introducer,
wherein the linear encoder sensor is positioned on or in the body of the attachment member in a position adjacent to the attachment member lumen.

10. The system of claim 9, wherein the proximal hub includes a hemostasis valve, such that when the attachment member is coupled to the proximal hub, the hemostasis valve is positioned distal to the linear encoder sensor and proximal to the introducer catheter.

11. The system of claim 1, wherein the linear encoder sensor is positioned on a printed circuit board assembly (“PCBA”), a microcontroller being positioned on the PCBA so that the signal generated by the linear encoder sensor is capable of passing to the microcontroller.

12. The system of claim 11, wherein a signal amplifier is positioned on the PCBA so that the signal generated by the linear encoder sensor is capable of passing to the signal amplifier before passing to the microcontroller.

13. The system of claim 11, wherein a noise filter is positioned on the PCBA so that the signal generated by the linear encoder sensor is capable of passing to the noise filter before passing to the microcontroller.

14. The system of claim 11, wherein the microcontroller is operably coupled to a display device configured to display information indicative of the axial distance which the catheter has traveled relative to the introducer.

15. The system of claim 1, wherein the introducer includes a motorized actuator configured to contact the catheter when the catheter is positioned within the introducer, the actuator configured to advance the catheter distally through the introducer.

16. The system of claim 15, wherein the actuator is also configured to retract the catheter proximally through the introducer.

17. The system of claim 15, wherein the actuator is a wheel actuator configured to rotate to advance the catheter distally.

18. The system of claim 15, wherein the introducer includes at least one button that, upon being depressed, sends a signal to the actuator to advance the catheter distally through the introducer.

19. The system of claim 15, wherein the actuator is configured to receive a signal from the linear sensor encoder, and the actuator is configured to advance the catheter distally based, at least in part, on the signal received from the linear encoder sensor.

20. The system of claim 15, wherein the introducer includes an idler and a biasing mechanism operably coupled to the idler, the biasing mechanism imparting force on the idler to push the idler toward the actuator,
wherein when the catheter is positioned through the introducer, the catheter is in contact with both the actuator and the idler.
